(19) United States
(12) Patent Application Publication
(10) Pub. No.: US 2022/0380755 A1
(43) Pub. Date: Dec. 1, 2022

(54) DE-NOVO K-MER ASSOCIATIONS BETWEEN MOLECULAR STATES

(71) Applicant: JumpCode Genomics, Inc., San Diego, CA (US)

(72) Inventor: Keith Brown, Carlsbad, CA (US)

(21) Appl. No.: 17/770,803

(22) PCT Filed: Oct. 22, 2020
(86) PCT No.: PCT/US2020/056904

§ 371 Date: Apr. 21, 2022

Related U.S. Application Data
(60) Provisional application No. 62/924,590, filed on Oct. 22, 2019.

(51) Int. Cl. C12N 15/10; G16B 30/20; C12Q 1/6869; C12Q 1/6855; C12Q 1/6806

(52) U.S. Cl. CPC C12N 15/1096; G16B 30/20; C12Q 1/6869; C12Q 1/6855; C12Q 1/6806

(57) ABSTRACT

Provided are methods for preparation and analysis of nucleic acids. Some embodiments include reverse transcribing the RNA with barcoded primers to produce cDNA while maintaining the DNA in the sample, sequencing the DNA and cDNA together, and differentiating the sequenced DNA and cDNA using the barcode or barcodes of the primers. Some embodiments include analyzing the DNA and cDNA sequences of multiple samples separating reads into k-mers, and comparing the k-mers between samples to identify differential sequences between the sequences of the samples.

### CROSS REFERENCE

This application claims the benefit of U.S. Provisional Application No. 62/924,590, filed on Oct. 22, 2019, which is incorporated herein by reference in its entirety.

### BACKGROUND

The disclosure herein relates to the field of molecular biology, such as methods and compositions for preparation and analysis of nucleic acids. Specifically, the disclosure relates to methods and compositions for reverse transcribing the RNA with barcoded primers to produce cDNA while maintaining the DNA in the sample, sequencing the DNA and cDNA together, and differentiating the sequenced DNA and cDNA using the barcode or barcodes of the primers.

High throughput, massively parallel techniques (such as microarrays and sequencing) offer the simultaneous readout of many unique nucleic acid molecules from a sample. Most of these methods do not allow for the simultaneous interrogation of both RNA and DNA from a sample without having to split the sample and separately isolate the RNA and DNA molecules. If both RNA and DNA is interrogated simultaneously, the result will not enable a researcher to determine if the sequence information came from an RNA molecule or a DNA molecule. This is usually due to the fact that RNA molecules are converted into a more stable cDNA molecule before sequencing or hybridization, rendering the molecule source as indeterminate.

### SUMMARY

Disclosed herein, in some embodiments, are barcoding library preparation schemes for simultaneously preparing sequencing libraries from both RNA and DNA templates. Rather than splitting samples, some embodiments include a single workflow that adds a barcode specific to RNA templates so that those RNA molecules can be differentiated from DNA molecules in a sequencing run. Some embodiments relate to a method, or a method of analyzing nucleic acid sequences comprising: providing a sample comprising DNA and RNA; reverse transcribing the RNA with a primer comprising a barcode to produce cDNA while maintaining the DNA in the sample; sequencing the DNA and the cDNA together; and differentiating the sequenced DNA and cDNA using the barcode or barcodes of the primers. In some embodiments, the barcoded primer comprise a random nucleic acid sequences. In some embodiments, the DNA is maintained in the sample by avoiding heating of the sample to denature the DNA prior to and during the reverse transcription of the RNA. Some embodiments further comprise fragmenting the DNA and RNA. Some embodiments further comprise tagmenting the DNA and cDNA. In some embodiments, the tagmentation comprises use of a transposase. In some embodiments, the transposase comprises a Tn5 transposase. In some embodiments, the transposase adds an adapter sequence to the DNA and cDNA. Some embodiments further comprise conducting end repair of the DNA and cDNA with a strand displacing polymerase. Some embodiments further comprise conducting A-tailing and adapter ligation to the DNA and/or cDNA. In some embodiments, the barcoded primers comprise an adapter sequence. Some embodiments further comprise adding a sample-specific index to the DNA and/or cDNA. Some embodiments further comprise determining a mutation in the DNA, and determining whether the RNA comprises the mutation. In some embodiments, the sample comprises a tumor or cancer sample. Some embodiments further comprise identifying a DNA pathogen in the sample and an RNA pathogen in the sample. In some embodiments, the DNA pathogen comprises a bacterium, fungus, or virus. In some embodiments, the RNA pathogen comprises a virus. Some embodiments further comprise identifying a microbe in the sample based on the sequenced DNA, and identifying whether the microbe is alive or dead based on the sequenced RNA or cDNA.

Disclosed herein, in some embodiments, are k-mer based statistical analyses showing presence/absence or increase/decrease in k-mers between two sample types. Some embodiments relate to a method for analysis of nucleic acid sequences, comprising: providing nucleic acid sequence reads for each of at least two samples—a first sample and a second sample; separating the reads of each sample into k-mers; comparing the k-mers of the first sample of the at least two samples to the k mers of the second sample of the at least two samples; identifying a statistical difference between the k-mers of the first and second samples, thereby identifying a differential sequence between the reads the first and second samples. In some embodiments, the each of k-mers comprises a sequence length of about 10, 25, 50, 75, 100, 125, 150, 250, or a range defined by any two of the aforementioned integers, or more, nucleotides. Some embodiments further comprise performing a local de novo assembly to expand a length of a differential sequence. Some embodiments further comprise identifying a genome region associated with the differential sequence. In some embodiments, the nucleic acid sequence reads are provided by a method that includes sequencing DNA and cDNA together as described in some embodiments herein.

Disclosed herein, in some embodiments, is a method for sequencing analysis, comprising: simultaneously sequencing RNA and DNA in a sample without separately isolating the RNA and DNA from the sample; determining a mutation in the DNA; and determining whether the RNA comprises the mutation. In some embodiments, the sample is a tumor or cancer sample. In some embodiments, the RNA and DNA are prepared and sequenced together using a method as described herein.

Disclosed herein, in some embodiments, is a method for pathogen sequencing analysis, comprising: simultaneously sequencing RNA and DNA in a sample without separately isolating the RNA and DNA from the sample; and based on the simultaneously sequenced DNA and RNA, identifying a DNA pathogen in the sample and an RNA pathogen in the sample. In some embodiments, the DNA pathogen comprises a bacterium, fungus, or virus. In some embodiments, the RNA pathogen comprises a virus. In some embodiments, the RNA and DNA are prepared and sequenced together using a method as described herein.

Disclosed herein, in some embodiments, is a method for microbiome sequencing analysis, comprising: simultaneously sequencing RNA and DNA in a sample without separately isolating the RNA and DNA from the sample; identifying a microbe in the sample based on the sequenced DNA; and identifying whether the microbe is alive or dead based on the sequenced RNA. In some embodiments, the RNA and DNA are prepared and sequenced together using a method as described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic of a workflow for a method, in accordance with some embodiments described herein.

FIG. 2 is a schematic showing an example sample preparation method using tagmentation.

FIG. 3 is a schematic showing an example sample preparation method using tagmentation and adapters.

FIG. 4 is a schematic showing an example sample preparation method where DNA and RNA from a sample are fragmented together.

FIG. 5 shows an image including a representation of nucleic acids, and a method of differentiating DNA and RNA sequences.

FIG. 6 shows an image including a representation of nucleic acid sequence reads and sequence assembly.

### DETAILED DESCRIPTION

Provided herein is a method for preparing simultaneously from a biological sample, such as an isolated cell or tissue, a nucleic acid sample comprising both DNA and RNA. Further provided herein is a method for analyzing the nucleic acid sample for the DNA as well as the RNA, wherein the analysis allows for identification of the source molecule, whether it is the DNA or the RNA. Numerous applications would benefit from the ability to prepare samples containing both RNA and DNA simultaneously as well as to enable the identification of the source molecule as RNA or DNA. For example, in the sequencing analysis of a tumor (whether single cell or tumor tissue), the method described herein can allow for determining the somatic mutation in the DNA from the tumor, while also determining whether the mutation is expressed and/or transcribed into RNA in the same cell. In an infectious disease study, one would want the ability to simultaneously detect RNA viruses as well as DNA pathogens such as bacteria or fungi. In a microbiome analysis, one would want the ability to detect all species of micro-organism while also determining if the organisms are alive or dead, and not simply present in the sample. The latter case being after anti-biotic treatment where the antibiotic may kill the bacteria, but the dead bacteria DNA would still be present in the sample as RNA typically degrades faster. Conversely, in some embodiments, a stable RNA from a certain virus may persist in the infected cell or tissue sample, whereas a relevant DNA signature may be helpful to determine whether replication-competent infective viral particles still persist or not. These approaches could be done over time points as well to see an increase in the organisms presence or an increase in cell specific mutation, while also determining if the RNA expression of these events are increasing or decreasing over time.

Disclosed herein is a novel library construction technology that allows for the simultaneous preparation of RNA and DNA molecules from a single sample source (e.g., a biological sample, such as a cell or a tissue or an acellular DNA/RNA comprising biological fluid) in a manner that enables an operator to determine whether the output (e.g., the nucleic acid sequence) is derived from an RNA or DNA molecule. Albeit, the method allows for an extraction of or the identification of an RNA or a DNA from the single sample. In addition, described herein include two novel analysis approaches for cancer and infectious disease testing.

Low pass sequencing has been used to replace microarrays for genotyping. Rather than building and assembling a genome from bottom up, sequencing reads are used against a database of haplotypes to determine the most likely haplotype from a given sample, and all genotypes in that haplotype are assigned. All that is needed is a reference genome to map the reads and a database of haplotypes. One problem is that not all species have a reference genome and therefore a database of variants. This issue came about when performing a small scale study on deaf cats. So rather than having to map reads to a reference, the reads were broken up into k-mers, and any statistical differences between the deaf cats and the non-deaf cats was looked for. Once those k-mers were identified to be associated with disease above a statistical threshold, a local de novo assembly was performed to expand the length of the differential sequence, and the region of the genome that appears to be the cause of the disease was identified.

The methods and compositions described herein can also be applied in cancer diagnosis. The way sequencing is done today in cancer is to take a tumor sample and compare it to the matched normal sample (usually blood) from the same individual. Both samples are sequenced to high coverage, reads are mapped to a reference, and mutations are identified by finding those found in the tumor and not in the normal sample. This approach including the mapping and assembly is expensive and also means that any dramatic differences in the genomic sequence from the tumor are less likely to be mapped to the normal (healthy) human reference genome. If there are mobile element rearrangements or viral insertions, those sequence reads get thrown away because they do not map to the reference. So it was determined that any significant differences between the sequence of the tumor and the normal sample could be identified by simply comparing the k-mers from the sequencing data and looking to see if there is a statistical threshold that could show a difference between the sample types, without the negative weighting of sequencing reads that are required, in some embodiments, to be mapped to a reference. This would also enable looking at viral insertions or any major changes. It can also be done with much less sequencer capacity than the 100× coverage that is needed to detect a mutation in a heterogeneous sample.

The methods and compositions described herein can also be used to analyzing both RNA and DNA within the samples. In the case of oncology, this is an unbiased approach to look at genomic alterations and determine if they are transcribed, and in the case of infectious disease, to determine the presence or absence of any viral, fungal or bacterial pathogens between healthy and sick individuals, or for example the same individual who goes to the hospital on day one and again on the day they leave.

### Definitions

A partial list of definitions is as follows.

“Amplified nucleic acid” or “amplified polynucleotide” is any nucleic acid or polynucleotide molecule whose amount has been increased at least two fold by any nucleic acid amplification or replication method performed in vitro as compared to its starting amount. For example, an amplified nucleic acid is obtained from a polymerase chain reaction (PCR) which can, in some instances, amplify DNA in an exponential manner (for example, amplification to 2ⁿ copies in n cycles). Amplified nucleic acid can also be obtained from a linear amplification.

“Amplification product” can refer to a product resulting from an amplification reaction such as a polymerase chain reaction.

An “amplicon” is a polynucleotide or nucleic acid that is the source and/or product of natural or artificial amplification or replication events.

The term “biological sample” or “sample” generally refers to a sample or part isolated from a biological entity. The biological sample may show the nature of the whole and examples include, without limitation, bodily fluids, dissociated tumor specimens, cultured cells, and any combination thereof. Biological samples can come from one or more individuals. One or more biological samples can come from the same individual. One non limiting example would be if one sample came from an individual's blood and a second sample came from an individual's tumor biopsy. Examples of biological samples can include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. The samples may include nasopharyngeal wash. Examples of tissue samples of the subject may include but are not limited to, connective tissue, muscle tissue, nervous tissue, epithelial tissue, cartilage, cancerous or tumor sample, or bone. The sample may be provided from a human or animal. The sample may be provided from a mammal, including vertebrates, such as murines, simians, humans, farm animals, sport animals, or pets. The sample may be collected from a living or dead subject. The sample may be collected fresh from a subject or may have undergone some form of pre-processing, storage, or transport.

“Bodily fluid” generally can describe a fluid or secretion originating from the body of a subject. In some instances, bodily fluids are a mixture of more than one type of bodily fluid mixed together. Some non-limiting examples of bodily fluids are: blood, urine, bone marrow, spinal fluid, pleural fluid, lymphatic fluid, amniotic fluid, ascites, sputum, or a combination thereof.

“Complementary” or “complementarity” can refer to nucleic acid molecules that are related by base-pairing. Complementary nucleotides are, generally, A and T (or A and U), or C and G (or G and U). Two single stranded RNA or DNA molecules are said to be substantially complementary when the nucleotides of one strand, optimally aligned and with appropriate nucleotide insertions or deletions, pair with at least about 90% to about 95% complementarity, and more preferably from about 98% to about 100%) complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions. Hybridization temperatures are generally at least about 2° C. to about 6° C. lower than melting temperatures (Tₘ).

A “barcode” or “molecular barcode” is a material for labeling. The barcode can label a molecule such as a nucleic acid or a polypeptide. The material for labeling is associated with information. A barcode is called a sequence identifier (i.e. a sequence-based barcode or sequence index). A barcode is a particular nucleotide sequence. A barcode is used as an identifier. A barcode is a different size molecule or different ending points of the same molecule. Barcodes can include a specific sequence within the molecule and a different ending sequence. For example, a molecule that is amplified from the same primer and has 25 nucleotide positions is different than a molecule that is amplified and has 27 nucleotide positions. The addition of positions in the 27-mer sequence is considered a barcode. A barcode is incorporated into a polynucleotide. A barcode is incorporated into a polynucleotide by many methods. Some non-limiting methods for incorporating a barcode can include molecular biology methods. Some non-limiting examples of molecular biology methods to incorporate a barcode are through primers (e.g., tailed primer elongation), probes (i.e., elongation with ligation to a probe), or ligation (i.e., ligation of known sequence to a molecule).

A barcode is incorporated into any region of a polynucleotide. The region is known. The region is unknown. The barcode is added to any position along the polynucleotide. The barcode is added to the 5′ end of a polynucleotide. The barcode is added to the 3′ end of the polynucleotide. The barcode is added in between the 5′ and 3′ end of a polynucleotide. A barcode is added with one or more other known sequences. One non-limiting example is the addition of a barcode with a sequence adapter.

A barcode is associated with information. Some non-limiting examples of the type of information a barcode is associated with information include: the source of a sample; the orientation of a sample; the region or container a sample was processed in; the adjacent polynucleotide; or any combination thereof.

In some cases, a bar code is made from combinations of sequences (different from combinatorial barcoding) and is used to identify a sample or a genomic coordinate and a different template molecule or single strand the molecular label and copy of the strand was obtained from. In some cases a sample identifier, a genomic coordinate and a specific label for each biological molecule may be amplified together. Barcodes, synthetic codes, or label information can also be obtained from the sequence context of the code (allowing for errors or error correcting), the length of the code, the orientation of the code, the position of the code within the molecule, and in combination with other natural or synthetic codes.

A barcode may be added before pooling of samples. When the sequences are determined of the pooled samples, the barcode is sequenced along with the rest of the polynucleotide. The barcode may be used to associate the sequenced fragment with the source of the sample.

A barcode can also be used to identify the strandedness of a sample. One or more barcodes is used together. Two or more barcodes is adjacent to one another, not adjacent to one another, or any combination thereof.

“Double-stranded” can refer to two polynucleotide strands that have annealed through complementary base-pairing.

“Known oligonucleotide sequence” or “known oligonucleotide” or “known sequence” can refer to a polynucleotide sequence that is known. A known oligonucleotide sequence can correspond to an oligonucleotide that has been designed, e.g., a universal primer for next generation sequencing platforms (e.g., Illumina, 454), a probe, an adaptor, a tag, a primer, a molecular barcode sequence, an identifier. A known sequence can comprise part of a primer. A known oligonucleotide sequence may not actually be known by a particular user but is constructively known, for example, by being stored as data which may be accessible by a computer. A known sequence may also be a trade secret that is actually unknown or a secret to one or more users but may be known by the entity who has designed a particular component of the experiment, kit, apparatus or software that the user is using.

A “k-mer” as used herein may refer to unique subsequences of a sequence of length k. K-mers are used in computational genomics to refer to nucleotides of any length, for example, could be 2, 3, 4, 5, 6, 7, 8 etc. nucleotides long, . . . up to the total number of nucleotides of the sequence. Usually, k-mer refers to all of a sequence's length k subsequences, for example, all possible k-mers of a sequence GTAGA would be individual nucleotides, G, T, A, G, A, or di-nucleotides, e.g., GT, TA, AG, GA and so on; or trinucleotides. e.g., GTA, TAG, AGA and so on; or tetranucleotides, e.g., GTAG, TAGA so on; or the sequence GTAGA.

“Library” can refer to a collection of nucleic acids. A library can be a genomic DNA library, cDNA library, a combination of genomic DNA/cDNA library, or a DNA/RNA hybrid library. A library can contain one or more target fragments. In some instances the target fragments are amplified nucleic acids. In other instances, the target fragments are nucleic acid that is not amplified. A library can contain nucleic acid that has one or more known oligonucleotide sequence(s) added to the 3′ end, the 5′ end or both the 3′ and 5′ end. The library may be prepared so that the fragments can contain a known oligonucleotide sequence that identifies the source of the library (e.g., a molecular identification barcode identifying a patient or DNA source). In some instances, two or more libraries are pooled to create a library pool. Kits may be commercially available, such as the Illumina NEXTERA kit (Illumina, San Diego, Calif.).

The term “melting temperature” or “Tₘ” commonly refers to the temperature at which a population of double-stranded nucleic acid molecules becomes half dissociated into single strands. Equations for calculating the Tₘ of nucleic acids are well known in the art. One equation that gives a simple estimate of the Tₘ value is as follows: Tₘ=81.5+16.6(log 10[Na⁺])0.41(%[G+C])−675/n−1.0 m, when a nucleic acid is in aqueous solution having cation concentrations of 0.5 M or less, the (G+C) content is between 30% and 70%, n is the number of bases, and m is the percentage of base pair mismatches (see, e.g., Sambrook J et al., Molecular Cloning, A Laboratory Manual, 3rd Ed., Cold Spring Harbor Laboratory Press (2001)). Other references can include more sophisticated computations, which take structural as well as sequence characteristics into account for the calculation of Tₘ.

“Nucleotide” can refer to a base-sugar-phosphate combination. Nucleotides are monomeric units of a nucleic acid sequence (e.g., DNA and RNA). The term nucleotide includes naturally and non-naturally occurring ribonucleoside triphosphates ATP, TTP, UTP, CTG, GTP, and ITP, for example and deoxyribonucleoside triphosphates such as dATP, dCTP, dITP, dUTP, dGTP, dTTP, or derivatives thereof. Such derivatives can include, for example, [aS]dATP, 7-deaza-dGTP and 7-deaza-dATP, and, for example, nucleotide derivatives that confer nuclease resistance on the nucleic acid molecule containing them. The term nucleotide as used herein also refers to dideoxyribonucleoside triphosphates (ddNTPs) and their derivatives. Illustrative examples of dideoxyribonucleoside triphosphates include, ddATP, ddCTP, ddGTP, ddITP, ddUTP, ddTTP, for example. Other ddNTPs are contemplated and consistent with the disclosure herein, such as dd (2-6 diamino) purine.

“Polymerase” can refer to an enzyme that links individual nucleotides together into a strand, using another strand as a template.

“Polymerase chain reaction” or “PCR” can refer to a technique for replicating a specific piece of selected DNA in vitro, even in the presence of excess non-specific DNA. Primers are added to the selected DNA, where the primers initiate the copying of the selected DNA using nucleotides and, typically, Taq polymerase or the like. By cycling the temperature, the selected DNA is repetitively denatured and copied. A single copy of the selected DNA, even if mixed in with other, random DNA, is amplified to obtain thousands, millions, or billions of replicates. The polymerase chain reaction is used to detect and measure very small amounts of DNA and to create customized pieces of DNA.

The terms “polynucleotides” and “oligonucleotides” may include but is not limited to various DNA, RNA molecules, derivatives or combination thereof. These may include species such as dNTPs, ddNTPs, 2-methyl NTPs, DNA, RNA, peptide nucleic acids, cDNA, dsDNA, ssDNA, plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA. “Oligonucleotides,” generally, are polynucleotides of a length suitable for use as primers, generally about 6-50 bases but with exceptions, particularly longer, being not uncommon.

A “primer” generally refers to an oligonucleotide used to prime nucleotide extension, ligation and/or synthesis, such as in the synthesis step of the polymerase chain reaction or in the primer extension techniques used in certain sequencing reactions. A primer may also be used in hybridization techniques as a means to provide complementarity of a locus to a capture oligonucleotide for detection of a specific nucleic acid region.

“Primer extension product” or “extension product” used interchangeably herein generally refer to the product resulting from a primer extension reaction using a contiguous polynucleotide as a template, and a complementary or partially complementary primer to the contiguous sequence.

“Sequencing,” “sequence determination,” and the like generally refers to any and all biochemical methods that may be used to determine the order of nucleotide bases in a nucleic acid.

A “sequence” as used herein refers to a series of ordered nucleic acid bases that reflects the relative order of adjacent nucleic acid bases in a nucleic acid molecule, and that can readily be identified specifically though not necessarily uniquely with that nucleic acid molecule. Generally, though not in all cases, a sequence requires a plurality of nucleic acid bases, such as 5 or more bases, to be informative although this number may vary by context. Thus a restriction endonuclease may be referred to as having a ‘sequence’ that it identifies and specifically cleaves even if this sequence is only four bases. A sequence need not ‘uniquely map’ to a fragment of a sample. However, in most cases a sequence must contain sufficient information to be informative as to its molecular source. The terms “reads” or “sequence reads” typically refer to sequencing results, comprising the nucleic acid or the amino acid sequence of the nucleic acid (DNA or RNA) and the protein respectively.

A “subject” generally refers to an organism that is currently living or an organism that at one time was living or an entity with a genome that can replicate. The methods, kits, and/or compositions of the disclosure is applied to one or more single-celled or multi-cellular subjects, including but not limited to microorganisms such as bacterium and yeast; insects including but not limited to flies, beetles, and bees; plants including but not limited to corn, wheat, seaweed or algae; and animals including, but not limited to: humans; laboratory animals such as mice, rats, monkeys, and chimpanzees; domestic animals such as dogs and cats; agricultural animals such as cows, horses, pigs, sheep, goats; and wild animals such as pandas, lions, tigers, bears, leopards, elephants, zebras, giraffes, gorillas, dolphins, and whales. The methods of this disclosure can also be applied to germs or infectious agents, such as viruses or virus particles or one or more cells that have been infected by one or more viruses.

A “support” is solid, semisolid, a bead, a surface. The support is mobile in a solution or is immobile.

The term “unique identifier” may include but is not limited to a molecular bar code, or a percentage of a nucleic acid in a mix, such as dUTP.

A “primer” as used herein refers to an oligonucleotide that anneals to a template molecule and provides a 3′ OH group from which template-directed nucleic acid synthesis can occur. Primers comprise unmodified deoxynucleic acids in many cases, but in some cases comprise alternate nucleic acids such as ribonucleic acids or modified nucleic acids such as 2′ methyl ribonucleic acids.

As used herein, a nucleic acid is double-stranded if it comprises hydrogen-bonded base pairings. Not all bases in the molecule need to be base-paired for the molecule to be referred to as double-stranded.

The term “about” as used herein in reference to a number refers to that number plus or minus up to 10% of that number. The term used in reference to a range refers to a range having a lower limit as much as 10% below the stated lower limit, and an upper number up to 10% above the stated limit.

In one aspect, the present disclosure provides a method for isolating and preparing for high a throughput analysis nucleic acid samples from a biological source, such as, cells, tissue, or bodily fluids. As used herein, the term “source” may be used interchangeably to designate a biological source, for example a tissue, cell or bodily fluid; or to designate an isolated nucleic acid source material, for example, the DNA or RNA. One of skill in the art should not have any difficulty in understanding, given the context of the occurrence of the term in the disclosure. The method steps described herein comprise, first, obtaining a nucleic acids from a biological sample. Source “samples” may be derived from single cells, blood, urine, CSF, saliva (etc), environmental samples from soil, water, air, or cell free nucleic acids. In some embodiments, cells may be lysed to obtain the nucleic acid. In some embodiments, proper isolation/purification and removal of contaminants or nucleases are performed when appropriate using suitable techniques well known to one of skill in the art.

Provided herein is a method of analyzing sequences, comprising: providing a sample comprising DNA and RNA; reverse transcribing the RNA with barcoded primers to produce cDNA while maintaining the DNA in the sample; sequencing the DNA and cDNA together; and differentiating the sequenced DNA and cDNA using the barcode or barcodes of the primers. In some embodiments, the barcoded primers comprise random sequences. In some embodiments, providing a sample comprising DNA and RNA includes isolating from a biological sample, the nucleic acid, comprising both the DNA and the RNA. In some embodiments, providing a sample comprising DNA and RNA comprises thawing a sample comprising the nucleic acid comprising the DNA and RNA. Isolating may comprise freeze thawing and/or lysing the cells by suitable mechanisms.

In some embodiments, the DNA is maintained in the sample by avoiding a condition of denaturing of DNA, including, but not limited to, heating of the sample to denature the DNA prior to and during the reverse transcription of the RNA, and/or by placing the DNA in a controlled condition of pH and/or ionic strength.

In some embodiments, the method further comprising fragmenting the DNA and RNA. Fragmenting the sample may comprise digesting the sample using enzymatic digestion, such as blunt end generating digestion. In some embodiments, the enzymatic digestion may comprise generating overhanging ends. Enzymatic digestion may comprise addition of enzymes such as nucleases, (e.g. endonucleases). In some embodiments, the fragmentation may comprise fragmenting the nucleic acid into about 50-100 base pair fragments, about 100-150 base pair fragments, about 150-200 base pair fragments, about 200-250 base pair fragments, about 250-300 base pair fragments, about 300-350 base pair fragments, about 350-400 base pair fragments, about 400-500 base pair fragments. In some embodiments, the fragmentation may comprise fragmenting the nucleic acid into 50-300 base pair fragments. In some embodiments the fragmentation may comprise fragmenting the nucleic acid into 100-500 base pair fragments.

In some embodiments, the method comprises cleaning/purifying the reaction mixture samples of residual enzymes, such as endonucleases, ligases etc. in between two method steps. In some embodiments, the method comprises deactivating of residual enzymes (e.g. stopping an enzymatic reaction by stop solutions e.g. EDTA solutions).

In some embodiments, the method further comprising tagmenting the DNA and/or the cDNA.

In some embodiments, the tagmentation comprises use of a transposase. In some embodiments, the transposase comprises a Tn5 transposase. In some embodiments, the transposase adds an adapter sequence to the DNA and/or the cDNA.

In some embodiments, the method further comprising conducting end repair of the DNA and/or cDNA with a strand displacing polymerase. In some embodiments, the method further comprises conducting A-tailing and adapter ligation to the DNA and/or cDNA.

In some embodiments, the barcoded primer comprise an adapter sequence. In some embodiments, the method further comprises adding a sample-specific index to the DNA and/or cDNA. In some embodiments, the method further comprises determining a mutation in the DNA, and determining whether the RNA comprises the mutation.

In some embodiments, the sample comprises a tumor or cancer sample.

In some embodiments, the method further comprises identifying a DNA pathogen in the sample and an RNA pathogen in the sample. In some embodiments, the DNA pathogen comprises a bacterium, fungus, or virus. In some embodiments, the RNA pathogen comprises a virus

In some embodiments, the method further comprises identifying a microbe in the sample based on the sequenced DNA, and identifying whether the microbe is alive or dead based on the sequenced RNA.

Provided herein is a method for analysis of nucleic acid sequences, comprising: providing nucleic acid sequence reads for each of at least two samples; separating the reads of each sample into k-mers; comparing the k-mers of a first sample of the at least two samples to the k-mers of a second sample of the at least two samples; identifying a statistical difference between the k-mers of the first and second samples, thereby identifying a differential sequence between the reads the first and second samples. In some embodiments, the k-mers each comprise a sequence length of 10, 25, 50, 75, 100, 125, 150, 250, or a range defined by any two of the aforementioned integers, or more, nucleotides. In some embodiments, the method further comprises performing a local de novo assembly to expand a length of a differential sequence. In some embodiments, the method further comprises identifying a genome region associated with the differential sequence. In some embodiments, the nucleic acid sequence reads are provided by a method that includes sequencing DNA and cDNA together. In some embodiments, the analysis comprises analyzing a DNA and an RNA in the nucleic acid simultaneously.

In one embodiment, the method steps comprise barcoding an isolated nucleic acid sample. In one embodiment, once nucleic acids have been obtained, RNA molecules are first “barcoded” through a random priming reaction. In some embodiments, the construct of the synthetic random primer may comprise or consist of a 5′ fixed sequence and a 3′ random sequence of desired length and GC content. In some embodiments, the 5′ fixed sequence may be used for functional purposes (such as hybridization) or for identification purposes. In the example described herein, the 5′ sequence of the synthetic random primer is used to identify an RNA molecule in a sample. A reverse transcriptase is used to prime from the 3′ end of the random synthetic primer and form a cDNA/RNA duplex. It is preferred, in some embodiments, that the sample is not heated prior to this step so that DNA in the sample remains double stranded. This may result in unlabeled DNA molecules and barcoded cDNA/RNA hybrid molecules. In a subsequent step, a transposase system (Tn5 for example) can be used to “tagment” all double stranded molecules within the sample. In some embodiments, the transpososome complex can comprise a single transposon, e.g., a Tn5 dimer sequence to barcode each sample in the reaction. In some embodiments, the transpososome complex can consist of a Tn5 dimer with a single transposon sequence to barcode each sample in the reaction. In some embodiments, double stranded DNA molecules would have the barcode sequence on both ends of each fragment attached to the 3′ ends of the DNA fragments. In some embodiments, the RNA molecules from the RNA/cDNA duplex would have a tagmentation derived barcode on the 5′ end of the RNA molecule and the random primed barcode on the 5′ end of the cDNA molecule in the RNA/cDNA hybrids. A polymerase with strand displacement activity would then be used to fill in the opposite strands on both the DNA and cDNA/RNA duplex molecules. In some embodiments, the constructs of these intermediate molecules would comprise or consist of double stranded, blunt end DNA molecules with identical barcode sequences on both ends and double stranded cDNA/RNA molecules with a tagmentation derived barcode on one end and a random primed barcode on the other. The blunt end products can then be A-tailed, and sequencing adapters (with optional sample specific barcodes) can be ligated and standard NGS sequencing is performed. After sequencing and de-multiplexing sample reads, DNA sequence is determined from molecules with dual unique barcodes derived from the tagmentation process while RNA derived molecule sequence is identified from sequence reads that have a tagmentation derived barcode on one end and a random primed derived barcode on the other.

Using the same workflow as above, in some embodiments, the random primer with molecular barcode used in the RT step can also include a universal adapter site on the 5′ end. Example primer: 5′-universal sequence-RNA barcode sequence-NNNNNNN-3′. In this approach, the transposon sequence can also be a universal adapter sequence. DNA molecules would result in 2 universal sequences from the tagmentation process on both ends of a double stranded molecule and the cDNA/RNA molecules would also have two universal adapter sequences on both ends with a RNA specific barcode on one end. Sample specific indices would be added during a subsequent PCR step during amplification from the universal adapter sequences on the library molecules.

Another approach, in some embodiments, can include fragmentation of both the RNA and DNA in the sample first. Then random prime the single stranded RNA products as previously described at a temperature that would not denature the double stranded DNA molecules. All fragmented products would be end repaired (with T4 pol for example) to create blunt end double stranded products of DNA and barcoded RNA/cDNA hybrids. Products would then be end repaired, A-tailed, and adapters specific to the NGS sequencing platform would be ligated and may include sample specific barcodes in the adapter sequences for multiplex sequencing.

In all examples of methods and compositions, sequence derived from DNA or RNA molecules can be determined from the RNA specific barcoded sequences.

In cancer studies, whether nucleic acids are derived from single cells or isolated tissue, the ability to detect mutations in the tumor and determine whether those mutations alter gene expression or are transcribed themselves may result in the ability to determine driver vs. passenger mutations for tumorigenesis, treatment regimens, and/or disease progression may result in better outcomes. For infectious disease and microbiome analysis, the ability to detect (and potentially quantify) all viral, bacterial and fungal species in a sample is desired as a single universal infectious disease test. Particularly when combined with an effective host removal of over-abundant nucleic acids.

Such mutation analysis in cancer tissues and/or infectious disease and microbiome analysis have certain technical disadvantages. For example, while the infectious disease application uses k-mers from the sequence data to look up those sequences across a database of all known organisms, a thorough and complete analysis would require a full understanding of the microbial species that exist in the world, which is currently unavailable. In the first study from the human microbiome project, there were over 150,000 new species of bacteria identified. Most if not all of which have no known pathogenic properties. As these novel species did not exist in a database previously, a k-mer look up against a known database would not enable identification of the organisms in the sample.

As for the cancer application, such approach has disadvantage that short sequence reads are typically mapped to a healthy reference genome. By definition, the further the distance from the “healthy” reference that a short sequence read is, the lower the probability of mapping those reads to the reference can be achieved. In addition, viral insertion sequences or somatic mobile elements would be missed by the reference mapping approach, even at the enormously high coverage that is recommended for tumor sequencing.

To overcome such disadvantages, a reference free, un-biased k-mer based statistical association with low coverage sequencing as presented herein can be applied. The criteria for this analysis is at least two sample types for comparison. This could be a tumor and matched normal sample from the same individual, a healthy and sick (infectious disease) sample (including when they first enter a hospital and when they leave), or multiple time points from the same individual whether in regards to cancer or infectious disease. In the case of population or family based studies, it would be a simple statistical comparison of disease cases with the non-disease matched controls. Sequence reads are broken up into k-mers of variable length (say 20, 30, 50, 150 or longer) and a simple statistical association between the two sample types is performed. A statistical increase (or decrease) in k-mer sequence between the two (or more) sample types is performed to identify core sequences that differentiate the two conditions. Once the core k-mer sequence is determined, a local de-novo assembly is performed using the additional sequencing reads with high overlap. In some embodiments, it is preferred to have random start/stop points of sequence molecules to expand the k-mer length to a maximum. Once these extended sequences are determined, they can be queried through programs like BLAST to determine similarity to all known sequences. Rather than deep sequence coverage to build an assembly from the bottom up, this approach requires, in some embodiments, just enough sequencing depth to determine a statistical difference between sequence motifs of comparable sample types.

Therefore proposed herein is a simple, streamlined and low cost workflow as described above and in the following examples that enables the identification of all differential sequence events including point mutations, structural rearrangements, mobile element insertions, presence of pathogenic organisms, retroviral events with an unbiased and reference free approach that requires, in some embodiments, no a priori hypothesis as to events that may cause disease.

Efficient adapter ligation, tagmentation: Briefly, in exemplary ligation methods, a blunt end-ligation may be performed to ligate adaptors on to DNA libraries. In some embodiments, a commercially available kit may be used. In some embodiments, an in-house manufacturing method may further be employed for example for scaling the preparation to optimum proportions. Typically, library preparation comprises the following steps: DNA fragmentation, end-polishing, adaptor ligation, size selection, and PCR amplification. DNA insert library is resuspended in nuclease free water (2 μM in ligatable ends, ˜60 ng/μl for 50 bp dsDNA fragments, ˜250 ng/μl for 200 bp fragments, or ˜1.3 μg/μl for 1,000 bp fragments); DNA adaptors are resuspended in nuclease-free water (10 μM, ˜300 ng/μl for 50 bp adaptors); High concentration T4 DNA Ligase may be used, with accompanying T4 ligase buffer; master mix and recommended materials for a kit (e.g., New England Biolabs, NEBNext Ultra™ II DNA Library Prep Kit for Illumina®, NEB #E7645). The ligation reaction is stopped with 50 mM EDTA. In some embodiments, single strand DNA adaptors may be used. A single strand adaptor is a double-stranded oligonucleotide with a 3′ overhang of 3 random nucleotides, which can be efficiently ligated to the 3′ end of single strand DNA by T4 DNA ligase. Tn5-based DNA tagmentation approach simplifies adaptor ligation in a library construction. In this approach, two mosaic end (ME) adaptors harboring the annealing sites of two primers are firstly complexed with a hyperactive derivative Tn5 transposase to form transposome, which then tagmented DNA into tagments with adaptors at their 5′ ends. The DNA tagments may then be amplified with PCR by using specific primers, which produces the DNA library compatible with massively parallel sequencing.

Tn5 transposase systems: Transposase (Tnp) Tn5 is a member of the RNase superfamily of proteins which includes retroviral integrases. Tn5 may be found in Shewanella and Escherichia bacteria. It may be commercially available as a kit for small scale use. Tn5 transposon codes for antibiotic resistance to kanamycin and other aminoglycoside antibiotics. Transposition works through a “cut-and-paste” mechanism. Tn5 excises itself from the donor DNA and inserts into a target sequence, creating a 9-bp duplication of the target. Tn5 is often utilized in genome sequencing for fragmentation of the DNA. In some embodiments, a hyperactive variant of the Tn5 transposase is used, that mediates the fragmentation of double-stranded DNA and ligates synthetic oligonucleotides at both ends in a 5-min reaction.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

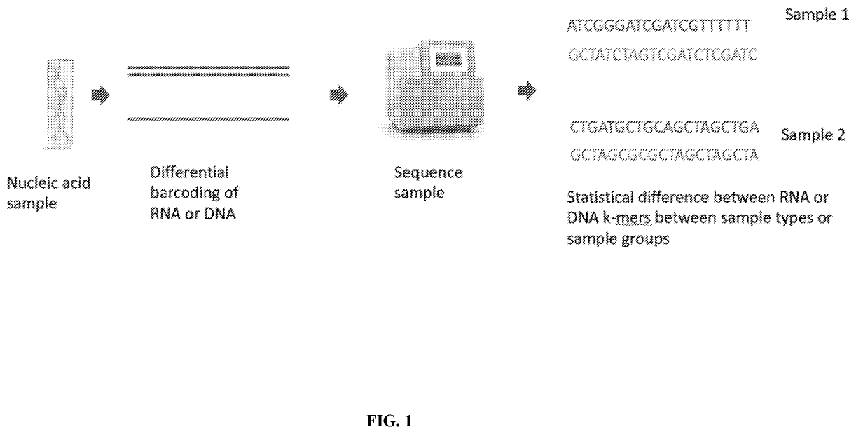

FIG. 1 shows a workflow of RNA/DNA sample preparation with RNA molecules uniquely barcoded and k-mer analysis. Once nucleic acids have been obtained, RNA molecules are first “barcoded” through a random priming reaction. The construct of the synthetic random primer may comprise or consist of a 5′ fixed sequence and a 3′ random sequence of desired length and GC content. The 5′ fixed sequence may be used for functional purposes (such as hybridization) or for identification purposes. In the example described here, the 5′ sequence of the synthetic random primer is used to identify an RNA molecule in a sample. A reverse transcriptase is used to prime from the 3′ end of the random synthetic primer and form a cDNA/RNA duplex. It is preferred, in some embodiments, that the sample is not heated prior to this step so that DNA in the sample remains double stranded. This results in unlabeled DNA molecules and barcoded cDNA/RNA hybrid molecules.

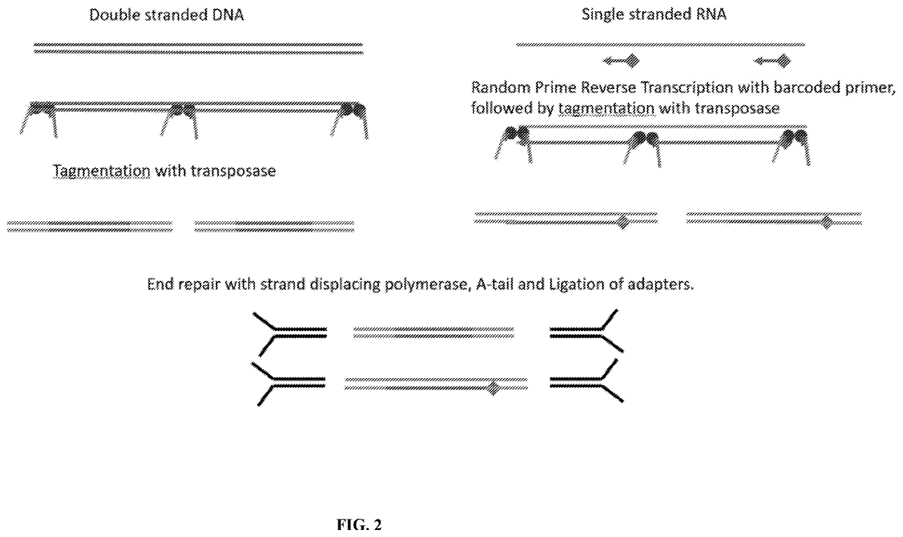

In a subsequent step, a transposase system (Tn5 for example) is used to “tagment” all double stranded molecules within the sample. FIG. 2 shows a schematic diagram of reverse transcription of RNA to make RNA/cDNA duplex (hybrid), followed by tagmentation of double stranded DNA and RNA/cDNA duplex, end repair with strand displacing polymerase, A-tail and ligation of sequencing. The transpososome complex in this example would comprise or consist of a Tn5 dimer with a single transposon sequence to barcode each sample in the reaction. Double stranded DNA molecules would have the barcode sequence on both ends of each fragment attached to the 3′ ends of the DNA fragments, whereas the RNA molecules from the RNA/cDNA duplex would have a tagmentation derived barcode on the 5′ end of the RNA molecule and the random primed barcode on the 5′ end of the cDNA molecule in the RNA/cDNA hybrids. A polymerase with strand displacement activity would then be used to fill in the opposite strands on both the DNA and cDNA/RNA duplex molecules. The constructs of these intermediate molecules would comprise or consist of double stranded, blunt end DNA molecules with identical barcode sequences on both ends and double stranded cDNA/RNA molecules with a tagmentation derived barcode on one end and a random primed barcode on the other. The blunt end products are then A-tailed, and sequencing adapters (with optional sample specific barcodes) are ligated and standard NGS sequencing is performed. After sequencing and de-multiplexing sample reads, DNA sequence is determined from molecules with dual unique barcodes derived from the tagmentation process while RNA derived molecule sequence is identified from sequence reads that have a tagmentation derived barcode on one end and a random primed derived barcode on the other.

Using the same workflow as above, the random primer with molecular barcode used in the RT step may also include a universal adapter site on the 5′ end. Example primer: 5′-universal sequence-RNA barcode sequence-NNNNNNN-3′. In this approach, the transposon sequence would also be a universal adapter sequence. DNA molecules would result in 2 universal sequences from the tagmentation process on both ends of a double stranded molecule and the cDNA/RNA molecules would also have two universal adapter sequences on both ends with a RNA specific barcode on one end. Sample specific indices would be added during a subsequent PCR step during amplification from the universal adapter sequences on the library molecules.

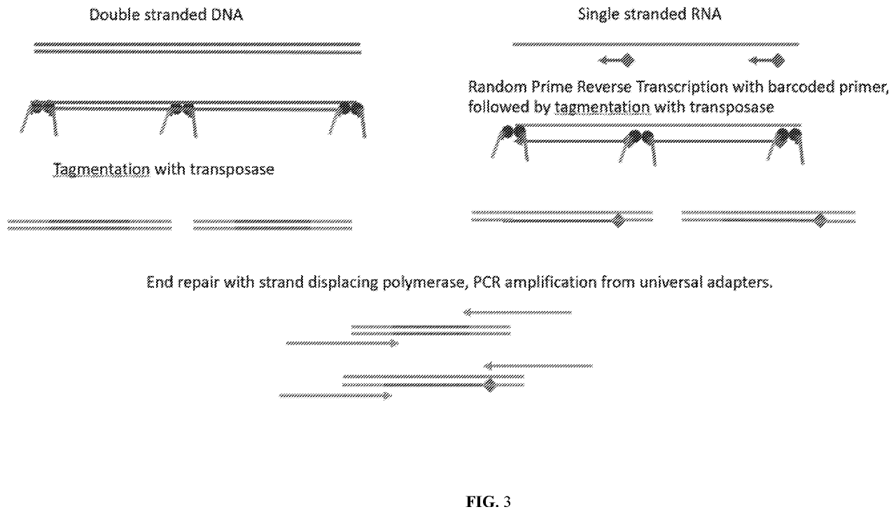

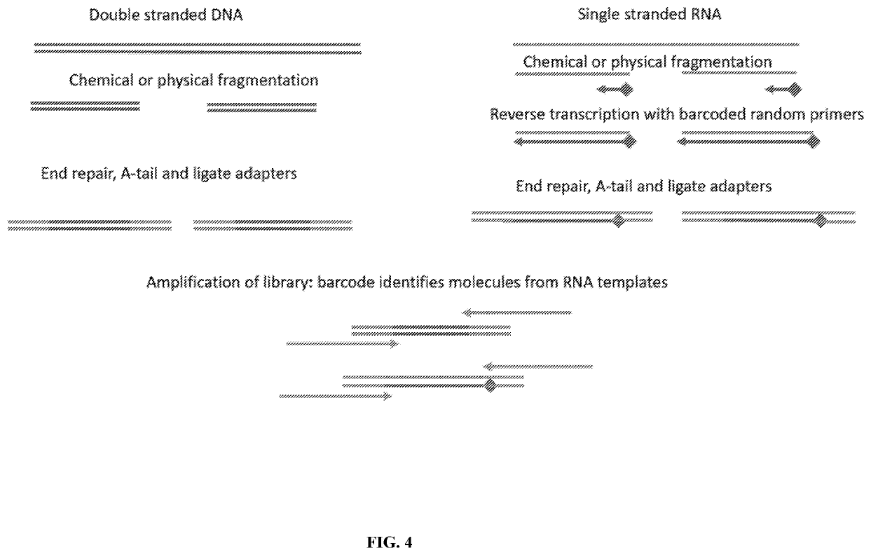

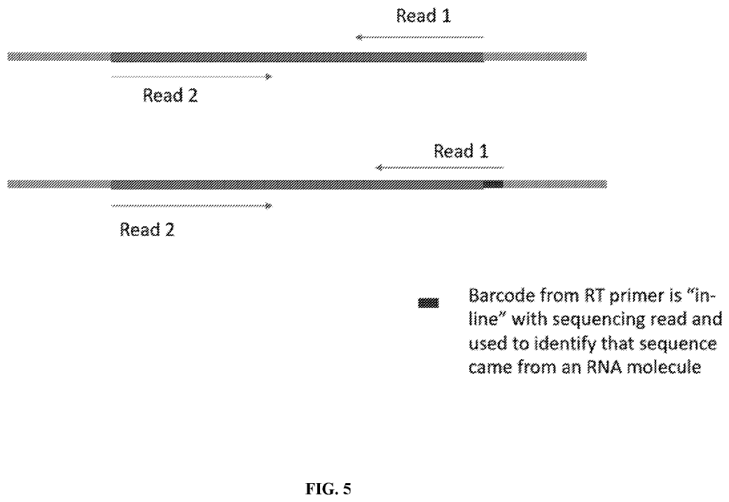

Another approach would be to fragment both the RNA and DNA in the sample first. Then random prime the single stranded RNA products as previously described at a temperature that would not denature the double stranded DNA molecules. All fragmented products would be end repaired (with T4 pol for example) to create blunt end double stranded products of DNA and barcoded RNA/cDNA hybrids. Products would then be end repaired, A-tailed, and adapters specific to the NGS sequencing platform would be ligated and may include sample specific barcodes in the adapter sequences for multiplex sequencing. FIG. 3 shows a schematic diagram of reverse transcription of RNA to make RNA/cDNA duplex (hybrid), followed by tagmentation of double stranded DNA and cDNA/RNA hybrid, end repair with strand displacing polymerase, and amplification of full length adapter sequences with sample specific barcodes. FIG. 4 shows a schematic diagram of fragmentation of RNA and DNA, followed by reverse transcription with barcoded random primers, and end repair, A-tail, and ligation of adaptors, and PCR amplification to incorporate sample specific barcodes. RNA derived sequencer reads have barcode. FIG. 5 shows a schematic diagram of simple read structure showing molecular barcode on RNA derived molecules.

In all examples of methods and compositions, sequence derived from DNA or RNA molecules can be determined from the RNA specific barcoded sequences.

### Example 2

In cancer studies, whether nucleic acids are derived from single cells or isolated tissue, the ability to detect mutations in the tumor AND determine whether those mutations alter gene expression or are transcribed themselves may result in the ability to determine driver vs. passenger mutations for tumorigenesis, treatment regimens, and/or disease progression may result in better outcomes.

For infectious disease and microbiome analysis, the ability to detect (and potentially quantify) all viral, bacterial and fungal species in a sample is desired as a single universal infectious disease test. Particularly when combined with an effective host removal of over-abundant nucleic acids.

Such mutation analysis in cancer tissues and/or infectious disease and microbiome analysis have certain technical disadvantages. For example, while the infectious disease application uses k-mers from the sequence data to look up those sequences across a database of all known organisms, a thorough and complete analysis would require a full understanding of the microbial species that exist in the world, which is currently unavailable or may not be readily available. In the first study from the human microbiome project, there were over 150,000 new species of bacteria identified. Most if not all of which have no known pathogenic properties. As these novel species did not exist in a database previously, a k-mer look up against a known database would not enable identification of the organisms in the sample.

As for the cancer application, such approach has disadvantage that short sequence reads are typically mapped to a healthy reference genome. By definition, the further the distance from the “healthy” reference that a short sequence read is, the lower the probability of mapping those reads to the reference can be achieved. In addition, viral insertion sequences or somatic mobile elements would be missed by the reference mapping approach, even at the enormously high coverage that is recommended for tumor sequencing.

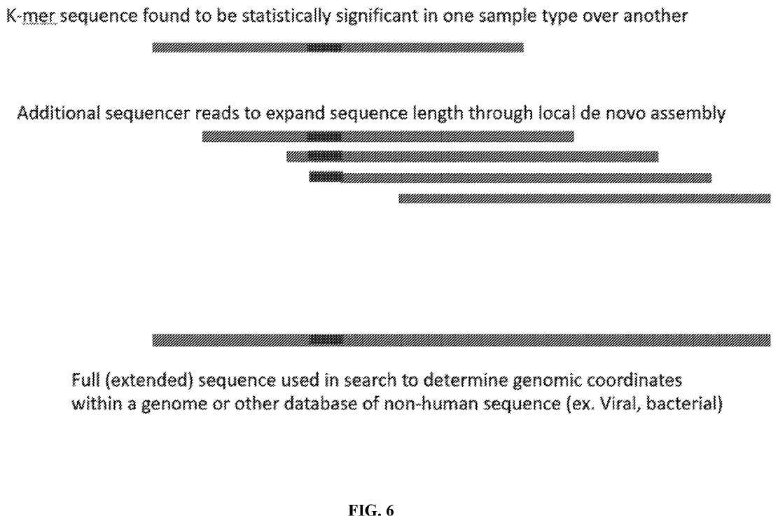

To overcome such problems, a reference free, un-biased k-mer based statistical association with low coverage sequencing as presented herein can be applied. The criteria for this analysis is at least two sample types for comparison. This could be a tumor and matched normal sample from the same individual, a healthy and sick (infectious disease) sample (including when they first enter a hospital and when they leave), or multiple time points from the same individual whether in regards to cancer or infectious disease. In the case of population or family based studies, it would be a simple statistical comparison of disease cases with the non-disease matched controls. Sequence reads are broken up into k-mers of variable length (say 20, 30, 50, 150 or longer) and a simple statistical association between the two sample types is performed. A statistical increase (or decrease) in k-mer sequence between the two (or more) sample types is performed to identify core sequences that differentiate the two conditions. Once the core k-mer sequence is determined, a local de-novo assembly is performed using the additional sequencing reads with high overlap. (Critical, in some embodiments, is random start/stop points of sequence molecules to expand the k-mer length to a maximum). Once these extended sequences are determined, they can be queried through programs like BLAST to determine similarity to all known sequences. Rather than deep sequence coverage to build an assembly from the bottom up, this approach requires, in some embodiments, just enough sequencing depth to determine a statistical difference between sequence motifs of comparable sample types. FIG. 6 shows a schematic diagram of constructing full (extended) sequence used in search to determine genomic coordinates within a genome (e.g., cancer mutation) or other database of non-human sequence (e.g., viral or bacterial sequences).

This simple, streamlined and low cost workflow enables the identification of all differential sequence events including point mutations, structural rearrangements, mobile element insertions, presence of pathogenic organisms, retroviral events with an unbiased and reference free approach that requires, in some embodiments, no a priori hypothesis as to events that may cause disease.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of analyzing nucleic acid sequences, comprising:
providing a sample comprising DNA and RNA;
reverse transcribing the RNA with a primer comprising a barcode to produce cDNA while maintaining the DNA in the sample;
sequencing the DNA and the cDNA together; and
differentiating the sequenced DNA and the sequenced cDNA using the barcode of the primers.

2. The method of claim 1, wherein the barcoded primer comprises a random sequence.

3. The method of claim 1 or 2, wherein the DNA is maintained in the sample by avoiding heating of the sample to denature the DNA prior to and during the reverse transcription of the RNA.

4. The method of any of claims 1-3, further comprising fragmenting the DNA and the RNA.

5. The method of any of claims 1-4, further comprising tagmenting the DNA and the cDNA.

6. The method of claim 5, wherein the tagmentation comprises use of a transposase.

7. The method of claim 6, wherein the transposase comprises a Tn5 transposase.

8. The method of claim 6 or 7, wherein the transposase adds an adapter sequence to the DNA and the cDNA.

9. The method of any of claims 1-8, further comprising conducting end repair of the DNA and the cDNA with a strand displacing polymerase.

10. The method of any of claims 1-9, further comprising conducting A-tailing and adapter ligation to the DNA and/or the cDNA.

11. The method of any of claims 1-10, wherein the barcoded primer comprises an adapter sequence.

12. The method of any of claims 1-11, further comprising adding a sample-specific index to the DNA and/or the cDNA.

13. The method of any of claims 1-12, further comprising determining a mutation in the DNA, and determining whether the RNA comprises the mutation.

14. The method of claim 13, wherein the sample comprises a tumor or cancer sample.

15. The method of any of claims 1-14, further comprising identifying a DNA pathogen in the sample and an RNA pathogen in the sample.

16. The method of claim 15, wherein the DNA pathogen comprises a bacterium, a fungus, or a virus.

17. The method of claim 15 or 16, wherein the RNA pathogen comprises a virus.

18. The method of any of claims 1-17, further comprising identifying a microbe in the sample based on the sequenced DNA, and identifying whether the microbe is alive or dead based on the sequenced cDNA.

19. A method for analysis of nucleic acid sequences, comprising:
providing nucleic acid sequence reads for a first sample and a second sample;
separating the reads of the first sample and the second sample into k-mers;
comparing the k-mers of the first sample to the k-mers of the second sample;
identifying a statistical difference between the k-mers of the first and second samples, thereby identifying a differential sequence between the reads the first and second samples.

20. The method of claim 19, wherein each of the k-mers comprises a sequence length of about 10, 25, 50, 75, 100, 125, 150, 250, or a range defined by any two of the aforementioned integers, or more, nucleotides.

21. The method of claim 19 or 20, further comprising performing a local de novo assembly to expand a length of a differential sequence.

22. The method of any of claims 19-21, further comprising identifying a genome region associated with the differential sequence.

23. The method of any of claims 19-22, wherein the nucleic acid sequence reads are provided by a method that includes sequencing DNA and cDNA together.

24. The method of any of claims 19-23, wherein the analysis comprises analyzing a DNA and an RNA in the first sample simultaneously.


SEQUENCE LISTING (4 sequences)

SEQ ID NO: 1 - DNA, 20 bp, Artificial Sequence
  Description of Artificial Sequence Synthetic oligonucleotide
atcgggatcgatcgtttttt

SEQ ID NO: 2 - DNA, 21 bp, Artificial Sequence
  Description of Artificial Sequence Synthetic oligonucleotide
gctatctagtcgatctcgatc

SEQ ID NO: 3 - DNA, 20 bp, Artificial Sequence
  Description of Artificial Sequence Synthetic oligonucleotide
ctgatgctgcagctagctga

SEQ ID NO: 4 - DNA, 20 bp, Artificial Sequence
  Description of Artificial Sequence Synthetic oligonucleotide
gctagcgcgctagctagcta